(19) United States
(12) Patent Application Publication
(10) Pub. No.: US 2025/0043305 A1
(43) Pub. Date: Feb. 6, 2025

(54) GENETIC CONSTRUCT FOR TRACKING AND/OR ABLATING QUIESCENT CELLS

(71) Applicant: UNIVERSITA' DEGLI STUDI DI TRENTO, TRENTO (IT)

(72) Inventors: Luca TIBERI, TRENTO (IT); Francesco ANTONICA, TRENTO (IT)

(73) Assignee: UNIVERSITA' DEGLI STUDI DI TRENTO, TRENTO (IT)

(21) Appl. No.: 18/705,193

(22) PCT Filed: Sep. 26, 2022
(86) PCT No.: PCT/IT2022/050256

§ 371 Date: Apr. 26, 2024

(30) Foreign Application Priority Data
Oct. 29, 2021 (IT) 102021000027851

(51) Int. Cl. C12N 15/85; C07K 14/72; C12N 9/12; C12N 15/62

(52) U.S. Cl. CPC C12N 15/85; C07K 14/721; C12N 9/1241; C12N 15/62; C07K 2319/55; C12Y 207/11022

(57) ABSTRACT

A genetic construct is described having a nucleotide sequence A (Cre+ERT2) with a nucleotide sequence SEQ ID NO. 1 coding for an enzyme recombinase Cre and a sequence SEQ ID NO. 2 coding for a mutated receptor for estrogen ERT2; or a nucleotide sequence A′ with a nucleotide sequence SEQ ID NO. 3 coding for the fragment A of the diphtheria toxin (DTA); and a nucleotide sequence B with a nucleotide sequence SEQ ID NO. 4 coding for the inhibitor of a mutant cyclin dependent kinase (CDK) p27K−.

### FIELD OF THE INVENTION

The present invention refers to genetic constructs and corresponding fusion proteins in addition to their related medical use. In particular, the genetic construct and its corresponding fusion protein can be used within the cell tracking and ablation, with particular reference to quiescent cells. The present invention also relates to a suitable pharmaceutical composition and a kit.

### BACKGROUND OF THE INVENTION

As well known, a cellular cycle is characterized by several phases (phase G1, phase S, phase G2 or phase M) and can be defined as a process in which a cell produced by a cell division is subjected to another cell division to produce a new cell. In past years, cellular research and studies have developed several techniques to characterize, analyse and monitor the various types of cells and their related cellular cycle. Moreover, the cellular cycle comprises a phase G0 which refers to a cellular state outside the replicative cellular cycle. In other words, it is stated that cells which stopped dividing themselves temporarily are in a quiescent state.

An example of a method to analyse a specific phase of the cellular cycle is the one based on the use of a BrdU label and that provides for the use of an anti-BrdU antibody (immune-histochemistry). However, this and other known methods alternative thereto, do not allow a real-time observation.

Moreover, in the prior art, no need is felt about finding techniques which allow a tracking and/or an ablation of the cellular type depending on the related proliferation/quiescence state.

Therefore, the need has been felt to find a solution which allows solving the above prior art problems.

### SUMMARY OF THE INVENTION

The Applicant has now found how to perform tracking and/or ablation of a cellular population, depending on the cellular proliferation/quiescence status. In particular, a genetic construct called CreERT2-p27K⁻ and a genetic construct called DTA-p27K⁻ have been found, whose products are not degraded by the quiescent cells, thereby allowing to be active only in the quiescent cells. In particular, the construct CreERT2-p27K⁻ can be used in the cellular tracking, while DTA-p27K can be used in cellular ablation.

Therefore, in a first aspect, the present invention deals with a genetic construct according to claim 1.

The invention allows tracking and/or ablating healthy or tumour quiescent cells, in particular stem cells. In a particularly advantageous aspect, the invention allowed dealing in depth with the role of tumour stem cells during progression and infiltration, always with particular reference to the sub-population of quiescent cells. Displaying of the quiescent cells only occurs due to the use of a fluorescent reporter and the global construct.

Tracking and ablating of cells in specific phases of the cellular cycle which can be obtained through the invention can be used in vivo, for example by creating transgenic animals, or through electroporation of cells as shown in the brain—in addition to in vitro transfection, for example of brain organoids.

Moreover, the construct of the invention, in addition to find application in relation to various types of cancer, can find application in various field of search, for example tissue development or regeneration, namely in those applications in which quiescent cells are deemed to be involved.

Further features and advantages of the invention will result from the description of example embodiments of the invention, provided as a description of the invention.

### BRIEF DESCRIPTION OF THE DRAWINGS

FIG. 1 shows a direct visualization of the cells Proml+ or Sox2+ slowly proliferating/quiescent in brain tumours of mice. a, Electroporation of cells in SVZ of P2 CD1 mice with pPB-CAG-TPR-MET-ires-mCherry and pPB-CAG-p53R273C-ires-mCherry (TP-Cherry) together with pPB-mProml-mVenus-p27K-(Proml-Venus-p27) or pPB-hSOX2-mVenus-p27K-(Sox2-Venus-p27). Mice have been sacrificed as soon as they show signs of pain or distress (human endpoint). b, Kaplan-Meier survival curve (p=0,0001) of mice injected only with TPR-MET (n=9 mice, black line) or TPR-MET and p53R273C (n=9 mice, blue line). c, Images representing a mosaic of brain sections which show the cells which express Proml-Venus-p27 inside the tumour (mCherry+). d, e, Representative images (d) and quantifications (e) of mVenus+ cells (green) which do not express Ki67 (grey) in core and marginal/infiltration regions of mCherry+ tumours (red) which express Proml-Venus-p27 (n=4 mice, 4818 cells) or Sox2-Venus-p27 (n=3 mice, 1217 cells). f, Images representing mVenus+ cells (green) which express N-cadherin (grey) in core or marginal/infiltration areas of mCherry+ tumours (red) which express Proml-Venus-p27. The level of expression of N-cadherin is show as multi-colour LUT. Asterisks refer to cells which express mVenus-p27. Scale (c) 2 mm, (d, f) 100 μm, (f, ROI) 40 μm. Data are presented as mean±s.e.m.

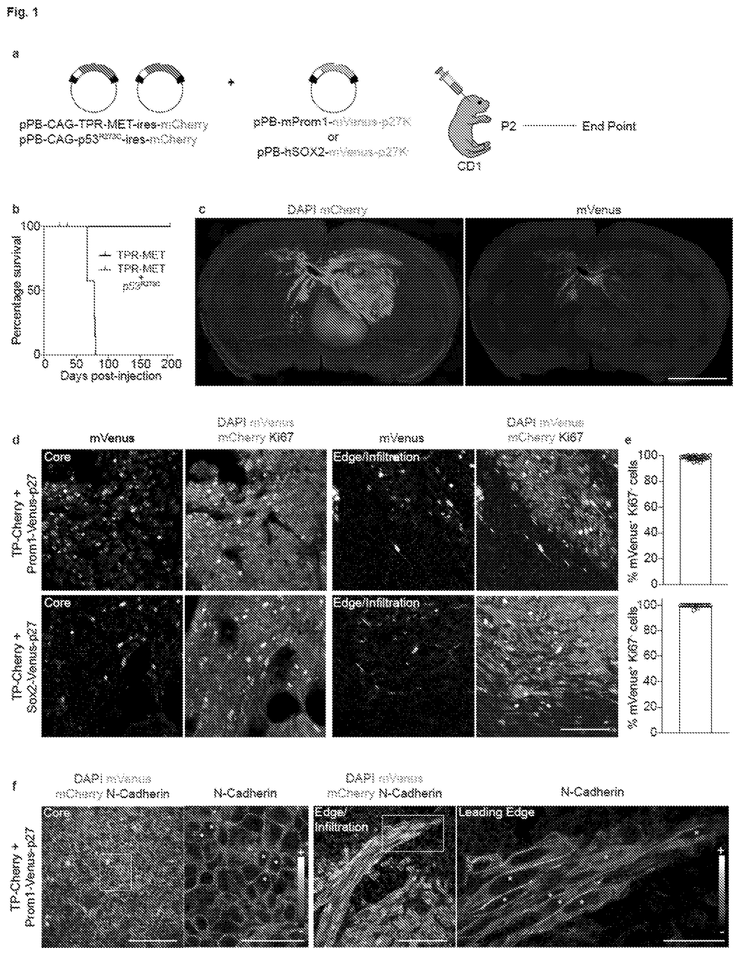

FIG. 2 show the tracking of the progeny of Proml+ cells, slowly proliferating/quiescent in tumours induced by TP after the treatment with Temozolomide. a, Electroporation of cells in SVZ of P2 CD1 mice with pPB-CAG-TPR-MET-ires-mCherry and pPB-CAG-p53R273C-ires-mCherry together with pPB-CAG-LSL-mVenus and pPB-mProml-CreERT2—p27K−. Mice have been injected with DMSO or Temozolomide (TMZ) every day from P30 to P34 and then with Tamoxifene every two days from P35 to P41. Brains have been sectioned at P42 and P56. b, Quantification of the tumour size shown as percentage of the area mCherry+ on the area DAPI+ in brain sections of mice treated with DMSO (n=7 mice a P42; n=3 mice at P56) or with TMZ (n=7 mice at P42; n=5 mice at P56). c, Quantification of infiltrating mVenus+ cells in the tumour of mice treated with DMSO (n=7 mice at P42; n=3 mice at P56) or with TMZ (n=7 mice at P42; n=5 mice at P56). d, e, Representative images (d) and quantifications (e) of mVenus (green) which expresses Ki67 (grey) in core or marginal/infiltration areas of mCherry+ tumours (red) of DMSO-(n=6 mice at P42, 576 cells for the core and 380 cells for the infiltrating edge; n=3 mice at P56, 219 cells for the core and 85 cells for the infiltrating edge) and treated with TMZ (n=7 mice at P42, 613 cells for the core and 1058 cells for the infiltrating edge; n=5 mice at P56, 952 cells for the core and 82 cells for the infiltrating edge). f, g, Representative images (f) and quantifications (g) of mVenus (green) which co-express OLIG2 (grey) in core or marginal/infiltration areas of mCherry+ tumours (red) of mice treated with DMSO (n=6 mice at P42, 736 cells for the core and 447 cells for the infiltrating edge; n=3 mice at P56, 237 cells for the core and 123 cells for the infiltrating edge) or with TMZ (n=7 mice at P42, 722 cells for the core and 527 cells for infiltrating edge; n=5 mice at P56, 1620 cells for the core and 229 cells for the infiltrating edge). Scale (d, f) 100 μm. Data are presented as mean±s.e.m. *P<0.05, **P<0.01 and ***P<0.001 calculated by the Kruskal-Wallis test followed by the Dunn test for the multiple comparison.

FIG. 3 shows the ablating of cells Proml+ slowly proliferating/quiescent, which reduces the infiltration of a TP-induced tumour. a, Electroporation of cells in the SVZ of P2 CD1 mice with pPB-CAG-TPR-MET-ires-mCherry, pPB-CAG-p53R273C-ires-mCherry and pPB-mProml-mVenus-p27K-with or without pPB-mProml-DTA-p27K−. Brains have been sectioned at P45. b, d, Representative images (b) and quantifications of the area mVenus+ (green) (c) and mCherry+ (red) (d) in brain sections of mice co-electroporated senza (n=4 mice) o with (n=5 mice) pPB-mProml-DTA-p27K−(Proml-DTA-p27). Every spot represents a section of the brain. e, Representative images of mCherry+ structures onco-stream situated on the infiltrating edge of the tumours. f, Quantification of the distance of the cells mCherry+ infiltrated from the tumour edge in the brain sections of mice co-electroporated without (n=4 mice) o with (n=5 mice) pPB-mProml-DTA-p27K-(Proml-DTA-p27). The 3 furthest cells for every section have been taken into account. g, Electroporation of cells in the SVZ of mice P2 CD1 with pPB-CAG-TPR-MET-ires-mCherry, pPB-CAG-p53R273C-ires-mCherry, pPB-mProml-mVenus-p27K−, pPB-mProml-CreERT2-p27K- and pPB-CAG-LSL-DTA. Mice have been injected with Tamoxifene every two days from P17 to P45. Brains have been selected at P45. h, j, Representative images (h) and quantifications of areas mVenus+ (green) (i) and mCherry+ (red) (j) in the brain sections of mice not injected (n=4) or injected with Tamoxifene (Tam) (n=4 mice). Every spot is a brain section. k, Quantification of the distance of the cells mCherry+ o mVenus+ infiltrated from the tumour edge in the brain sections of mice not injected (n=4 mice) or injected with Tamoxifene (n=4 mice). The 3 furthest cells for every section have been taken into account. Scale (b, h) 2 mm, (e) 200 μm. Data are presented as mean±s.e.m. (c, i, j) or median and quartile (f, k), **P<0.01, ***P<0.001 and ****P<0.0001, calculated by two-tail non-parametric Mann-Whitney tests.

FIG. 4 shows a direct visualization and live imaging of infiltrating slow-cycling cells in human brain cancer organoids. a, Schematic view of the differentiation protocol of pluripotent induced human stem cells in forebrain organoids. At day 35 the organoids have been electroporated with pPB-CAG-mCherry (Cherry) or pPB-CAG-TPR-MET-ires-mCherry and pPB-CAG-p53R273C-ires-mCherry (TP-Cherry) and cultivated for further 30 days (D35+ 30). b, Representative images of electroporated organoids at day 35+ 30. c, h, Representative images and quantifications of cells mCherry+ (red) which co-express Ki67 (c, d) or SOX2 (e, f) or NeuN (g, h) (grey) in organoids at D35+ 30 electroporated with Cherry or TP-Cherry (n=4-6 organoids, more than 300 or 1300 cells have been counted for each marker in electroporated organoids Cherry or TP-Cherry, respectively). i, Electroporation of organoids D35 with pPB-CAG-TPR-MET-ires-mCherry, pPB-CAG-p53R273C-ires-mCherry and pPB-CAG-mVenus-p27K-(TP-cherry/Venus-p27). The organoids have been fixed at D35+ 30. j, k, Representative images (j) and quantification (k) of cells mVenus+ (green) which do not express Ki67 (grey) in organoids TP-cherry/Venus-p27 at D35+ 30 (n=8 organoids, 298 cells). 1, Transplant of organoids TP-cherry/Venus-p27 in the brain of nude mice. Brains have been sectioned in the final huma spot. m, Representative image of the brain tumour derived from organoids TP-cherry/Venus-p27. n, o, Representative image (n) and quantification (o) of cells mCherry+ (red) and mVenus+ (green) which do not express Ki67 (grey) at the infiltrating edge of tumours derived from organoids (n=3 mice, 340 cells). p, Co-culture of TP-cherry/Venus-p27 and non-electroporated organoids D35+ 30. Organoids have been shot with a confocal microscope with rotary disks every 24 hours till infiltrating cells have been observed. q, Representative images of cells mCherry+ mVenus+ which are infiltrated in the host. An infiltrating cell is pointed out and every single reporter is shown. r, Representative image of the cryosection of the same organoids shown in p with cells mVenus+ (green) Cherry+ (red) Ki67—(grey) infiltrated in the host. Bars of the scale (b) 600 μm, (c, e, g, j, n) 100 μm, (m) 2 mm, (q) 200 μm and 50 μm (ROI), (r) 400 μm and 50 μm (ROI). Data are presented as mean±s.e.m, * P<0.05, ** P<0.01 calculated by the non-parametric, two-tail, unpaired Mann-Whitney test (d, f) or the two-tail Student test t (g).

FIG. 5 shows extended data of FIG. 1. Specificity test of mProml and hSOX2 promoters in mouse brains. a, Electroporation of cells in the SVZ of CD1 mice at P2 with pPB-CAG-mCherry and pPB-mProml-mVenus-p27K- or pPB-hSOX2-mVenus-p27K−. Brains have been sectioned at P30. b, Representative images of the expression of mVenus journalist under control of the Prom1 or Sox2 promoter. c, Electroporation of cells in the SVZ of P2 Prom1-CreERT2 mice with pPB-mProml-mCherry and pPB-CAG-LSL-mVenus. Mice have been injected daily with Tamoxifene from P26 to P29 and the brain has been sectioned at P30. d, Representative images (d) and quantification (e) of cells mCherry+ which express mVenus (n=3 mice, 213 cells). f, g, Representative images (f) and quantification (g) of cells mVenus+ which express SOX2 (n=2 mice, 36 cells). Scale (b) 400 μm, (d) 50 μm, (f) 100 μm and 30 μm (ROI). Data are presented as mean s.e.m.

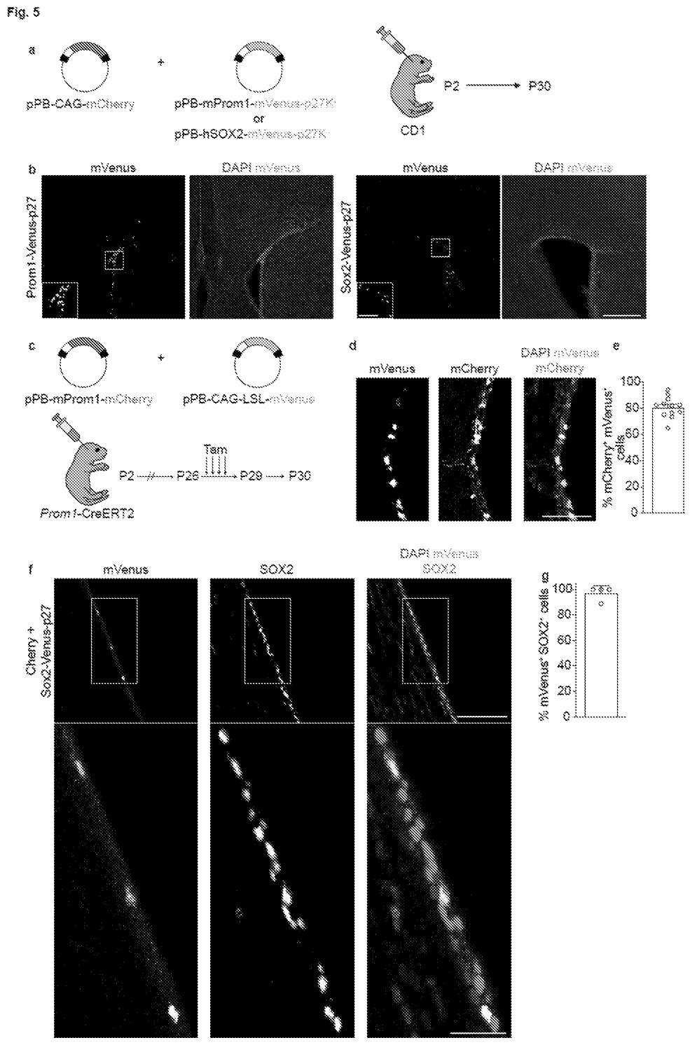

FIG. 6 shows extended data of FIG. 2. Specificity test of the cellular cycle of the reporter mVenus-p27K-in a mouse brain. a, Electroporation of cells in the SVZ of PCD1 mice at P2 with pPB-CAG-mCherry and pPB-mProml-mVenus-p27K- or pPB-hSOX2-mVenus-p27K−. Brains have been sectioned at P30. b, c, Representative images (b) and quantifications (c) of cells mVenus+ (green) which do not express Ki67 (grey) in the SVZ of mice which express Proml-Venus-p27 (n=3 mice, 171 cells) or Sox2-Venus-p27 (n=3 mice, 84 cells). d, Electroporation of cells in the SVZ of mice CD1 at P2 with pPB-CAG-mCherry and pPB-mProml-mVenus-p27K- or pPB-hSOX2-mVenus-p27K−. Mice have been injected with EdU at P29 and the brain has been sectioned at P30. e, f, Representative images (e) and quantifications (f) of cells mCherry+ (red) not labelled with EdU (grey) and/or which do not express mVenus (green) in the SVZ of mice which express Proml-Venus-p27 (n=4 mice, 104 cells) or Sox2-Venus-p27 (n=2 mice, 46 cells). g, Electroporation of cells in the SVZ of mice P2 CD1 with pPB-mProml-mVenus-p27K- or pPB-hSOX2-mVenus-p27K−. Mice have been injected with EdU at P23 and the brain has been sectioned at P30. h, i, Representative images (h) and quantifications (i) of cells mVenus+ (green) not labelled with EdU (grey) and/or which do not express Ki67 (red) in the SVZ of mice which express Proml-Venus-p27 (n=3 mice, 120 cells) or Sox2-Venus-p27 (n=3 mice, 97 cells). Scale (b) 100 μm, (e, h) 50 μm. Data are presented as mean±s.e.m.

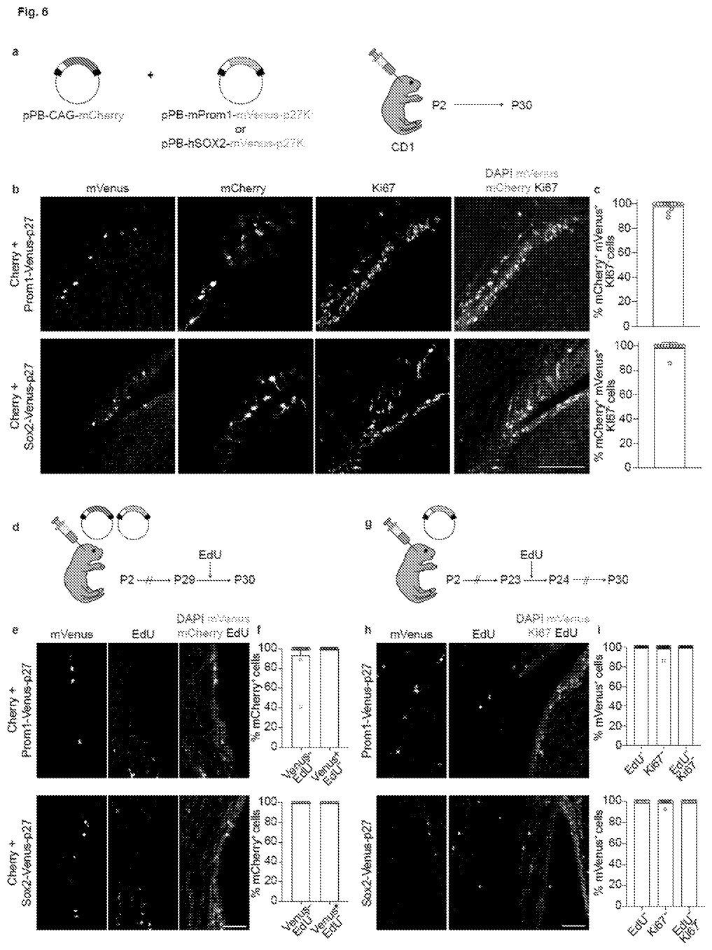

FIG. 7 shows extended data of FIG. 3. Characterization of tumours induced by TP and cells mVenus-p27K which express Prom1 or Sox2 in a mouse brain. a, b, RNA-seq analysis of TP-induced tumours and set of published data of mouse glioma and normal brain (ref 18 of the manuscript). Analysis of main components (PCA) (a) and hierarchical clustering (b) of TP-induced tumours and set of published data of RNA-seq of mouse glioma (CT2A, GL261, Mut3, 005) and normal brain. c, d, Representative images (c) and quantifications (d) of cells mVenus+ (green) which express SOX2 (grey) in the core and marginal/infiltration areas of mCherry+ (red) tumours which express Proml-Venus-p27 (n=3 mice, 1966 cells) or Sox2-Venus-p27 (n=3 mice, 627 cells). e, f, Representative images (and) and quantifications (f) of cells mVenus+ (green) which express OLIG2 (grey) in the core or in the infiltrating marginal areas of tumours mCherry+ (red) which express Proml-Venus-p27 (for core n=4 mice, 3762 cells; for infiltrating edge n=3 mice, 911 cells) or Sox2-Venus-p27 (for core n=3 mice, 907 cells; for infiltrating edge n=3 mice, 610 cells). Scale (c) 100 μm, (e) 100 μm and 50 μm (ROI). Data are presented as mean±s.e.m, *P<0.05, ****P<0.0001 calculated by non-parametric Mann-Whitney test (f).

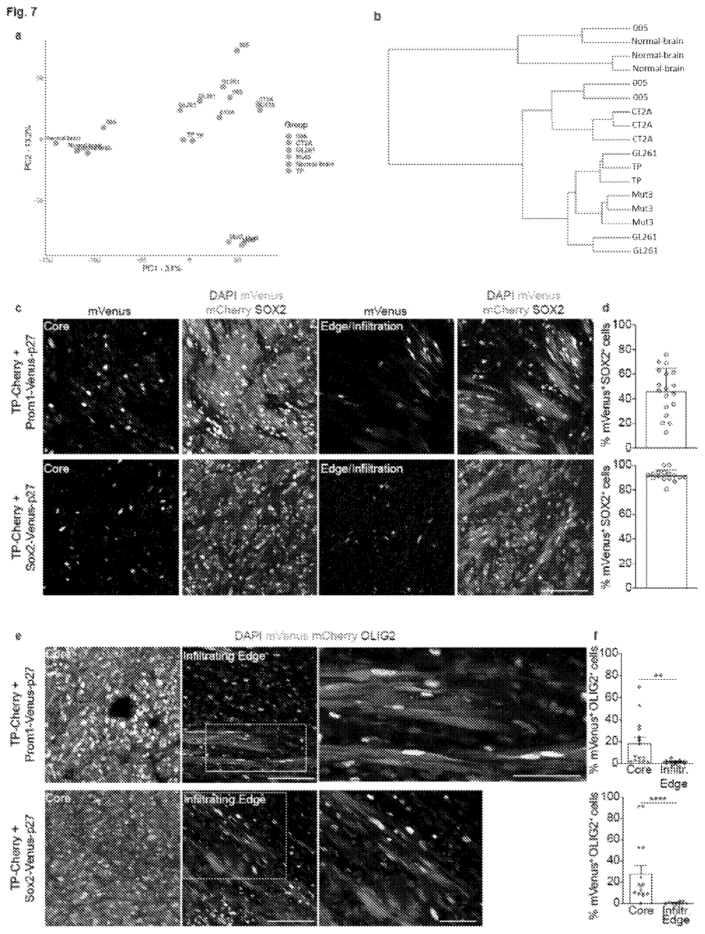

FIG. 8 shows extended data of FIG. 4. Specificity test of the cellular cycle of CreERT2-p27K-in a mouse brain. a, Electroporation of cells in the SVZ of CD1 mice at P2 with pPB-CAG-mCherry, pPB-CAG-LSL-mVenus and pPB-mProml-CreERT2-p27K−(Proml-CreER-p27). Mice have been injected with EdU+ Tamoxifene at P5 or with EdU at P4 followed by Tamoxifene at P5. All brains have been sectioned at P6. b, c, Representative images (b) and quantifications (c) of cells mVenus+ (green) not labelled with EdU (grey) in the SVZ of mice which express Proml-CreER-p27 and pPB-CAG-LSL-mVenus and injected with EdU+ Tamoxifene at P5 (n=4 mice, 127 cells) or EdU at P4 and Tamoxifene at P5 (n=3 mice, 173 cells). d, Electroporation of cells in the SVZ of P2 CD1 mice with pPB-CAG-mCherry, pPB-CAG-LSL-mVenus and pPB-mProml-CreERT2-p27K−. Mice have been injected daily with Tamoxifene from P25 to P28. All brains have been selected at P35. e, f, Representative images (e) and quantifications (f) of cells mVenus+ (green) which express SOX2 (grey) in the SVZ of mice injected with Tamoxifene (n=4 mice, 83 cells). Scale (b, and) 100 μm. Data are expressed as mean±s.e.m.

FIG. 9 shows extended data of FIG. 5. Tracking of the lineage of qProm1 cells in TP-induced tumours after treatment with TMZ of the mice. a, Representative images of cells mVenus+ (green) and mCherry+ (red) in sections of mouse brain not injected with Tamoxifene. b, Representative images of mVenus+ (green) and mCherry+ (red) in sections of mouse brain treated with DMSO or TMZ (and Tamoxifen) at P42 or P56. Scale (a, b) 2 mm.

FIG. 10 shows extended data of FIG. 6. Ablating of qProm1 and qSox2 in the SVZ of a mouse brain. a, Electroporation of cells in the SVZ of CD1 mice at P2 with pPB-CAG-mCherry, pPB-mProml-mVenus-p27K-with or without pPB-mProml-DTA-p27K−(Proml-DTA-p27). Brains have been sectioned at P30. b, c, Representative images (b) and quantifications (c) of cells mCherry+ (red) which express mVenus+ (green) in the SVZ of mice co-electroporated without (n=4 mice, 267 cells) or with (n=2 mice, 53 cells) Proml-DTA-p27. d, Electroporation of cells in the SVZ of mice P2 CD1 with pPB-CAG-mCherry, pPB-hSOX2-mVenus-p27K-with or without pPB-hSOX2-DTA-p27K-(Sox2-DTA-p27). Brains have been sectioned at P30. e, f, Representative images (e) and quantifications (f) of cells mCherry+ (red) which express mVenus+ (green) and SOX2 (grey) in the SVZ of mice co-electroporated without (n=3 mice, 107 cells) or with (n=3 mice, 74 cells) Sox2-DTA-p27. Scale (b, e) 100 μm. Data are presented as mean±s.e.m. ****P<0.0001 calculated through non-parametric Mann-Whitney test (c, f). Values of P are described in Supplementary statistical information.

FIG. 11 shows extended data of FIG. 7. Characterization of organoids of human brain cancer which express mVenus-p27K−. a, b, Representative images (a) and quantification (b) of cells mVenus+ (green) which express SOX2 (grey) in organoids TP-cherry/Venus-p27 at D35+ 30 (n=9 organoids, 364 cells). c, Representative images of the cryo-section of organoids co-cultivated with cells mVenus+ (green) Cherry+ (red) Ki67—(grey) infiltrated in the host. Scale (a) 100 μm, (c) 400 μm and 100 μm (ROI).

FIG. 12a shows the operating scheme of the tracking of specific quiescent cellular populations which can be obtained through the invention.

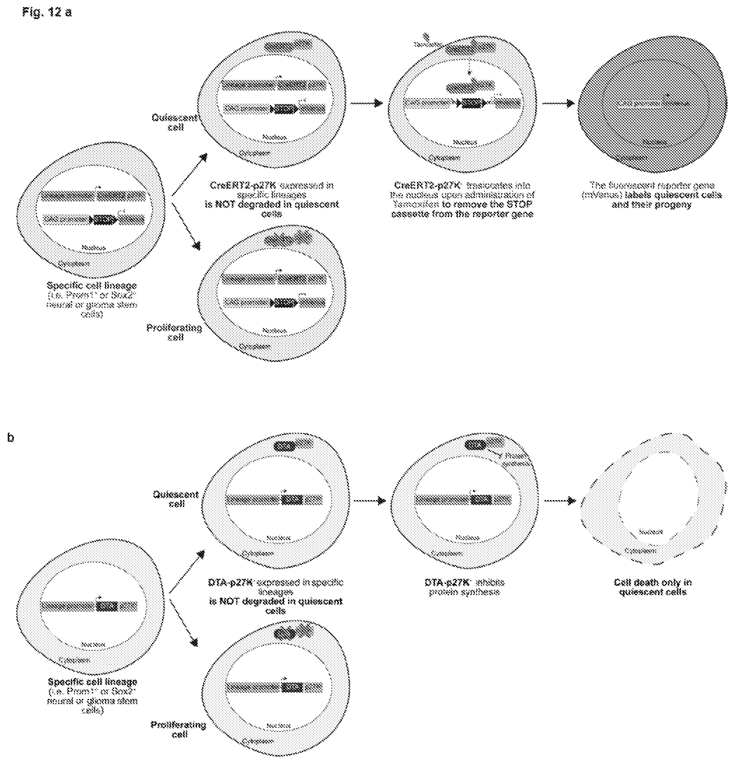

FIG. 12b shows the operating scheme of the ablating of specific quiescent cellular populations which can be obtained through the invention.

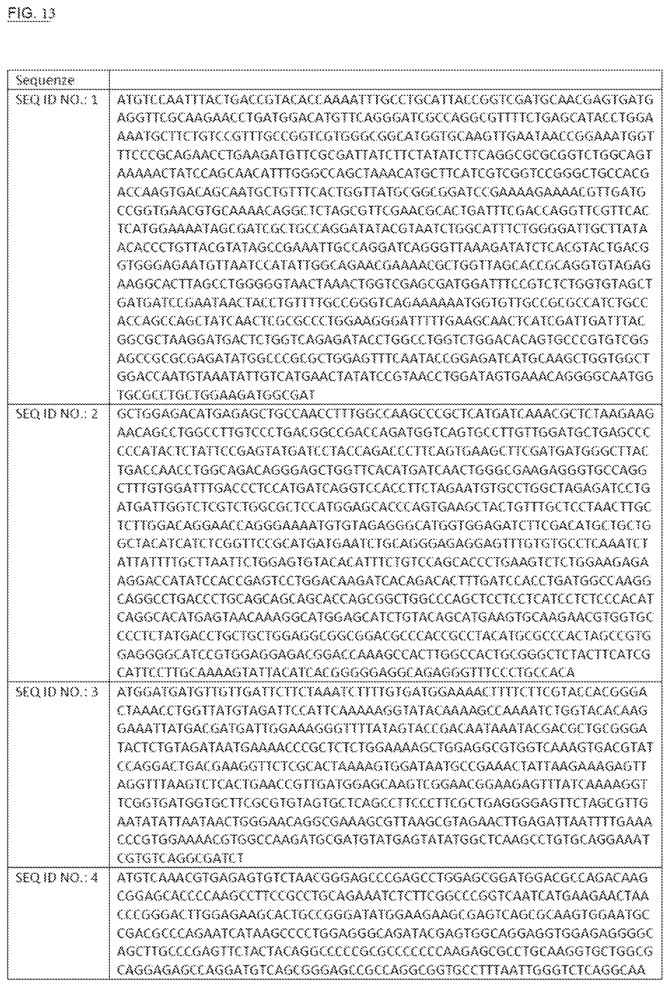

FIG. 13 shows a summarizing table of the sequences used in the present invention.

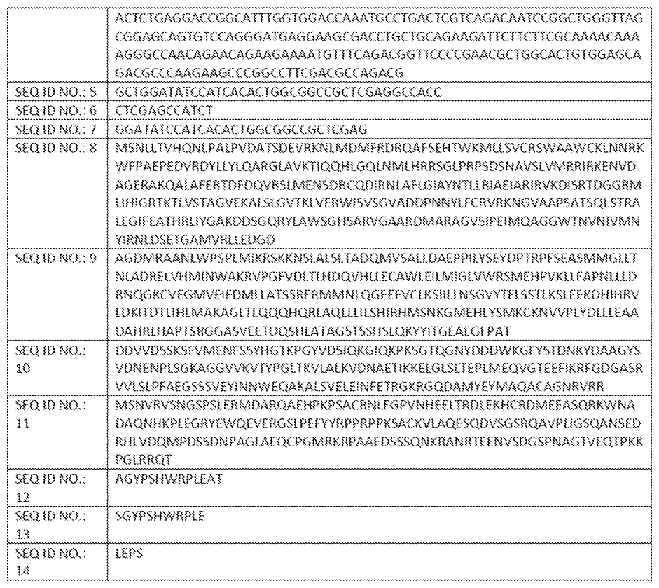

### DETAILED DESCRIPTION OF THE INVENTION

For the purposes of the invention, herein below definitions will be provided of some terms used in the present description and in the enclosed claims.

The term tracking means the activation of the transcription of a reporter system (for example coding gene for fluorescent protein) through the action of an enzyme called Cre recombinase which removes a gene cassette or sequence called Lox-stop-Lox this one responsible for blocking the expression of the reporter gene. The expression of the fluorescent reporter protein allows displaying/marking a certain cell and its progeny in time and in space. The expression of Cre in a certain cellular type (through a specific promoter) allows activating the tracking only in specific cellular types (for example, if the expression of Cre is activated through a promoter of the gene Prominin-1 or Sox2, it is possible to track the stem cells). If the Cre is fused ERT2, it allows its control and therefore activation only at the presence of Tamoxifene; CreERT2 in fact is expressed but not active. The activation can occur only through Tamoxifene, which, binding itself to ERT2, allows the Cre to perform its removal function of the cassette Los-stop-Lox. This allows activating the tracking system in specific cellular types (through a promoter) from a certain time (through Tamoxifene).

The term ablating or cellular removal means the activation or expression of a toxin (for example Toxin of the diphtheria—DTA) which induces inside the cell the inhibition of the protein synthesis which consequently brings about the cellular death. The expression of the DTA toxin in cellular sub-types and therefore the selective ablation of specific cellular types can be allowed through the use of specific promoters (for example Prominin-1 for stem cells). Moreover, the ablation can be induced in specific cellular types and starting from a certain time by combining the tracking system with ablating. If the expression of the DTA toxin is prevented by the cassette Lox-stop-Lox, only the combination with the system CreERT2 allows its activation in specific cellular typed (specific promoters which control CreERT2) and from a certain time (through treatment with Tamoxifene). In the present invention, as regards the tracking activity, it is performed through the construct CreERT2-p27K⁻ and related protein fusion product.

The genetic construct according to the invention codes for the enzyme inducible recombinase Cre (CreERT2) fused with p27K (mutant form lacking association sites to CDK), which requires the presence of Tamoxifene to have activity, can be obtained from the fusion of the sequence of the enzyme inducible recombinase Cre (CreERT2) with p27 (mutant form lacking association sites to CDK), where ERT2 is a mutated receptor for estrogen. Tamoxifene, as meant in all aspects described in the present document, can be provided through injection or supplemented to the cellular culture medium.

In the present invention, as regards the ablating activity, it is performed through the construct DTA-p27K and related protein fusion product.

Therefore, a first aspect of the present invention is a genetic construct comprising:
a nucleotide sequence A (Cre+ ERT2) comprising a nucleotide sequence SEQ ID NO. 1 coding for an enzyme recombinase Cre and a nucleotide sequence SEQ ID NO. 2 coding for a mutated receptor for estrogen ERT2; or
a nucleotide sequence A′ comprising a nucleotide sequence SEQ ID NO. 3 coding for the fragment A of the diphtheria toxin (DTA); and comprising
a nucleotide sequence B comprising a nucleotide sequence SEQ ID NO. 4 coding for the inhibitor of a mutant cyclin dependent kinase (CDK) p27K.

The above nucleotide sequences A or A′ and B can be connected by a linker sequence. Similarly, the sequences composing the nucleotide sequence A can be connected by a linker sequence.

The genetic construct can further comprise a linker sequence which mutually binds the nucleotide sequences A and B, wherein the linker sequence is preferably the sequence SEQ ID NO 5 (gctggatatccatcacactggcggccgctcgaggccacc).

The genetic construct can further comprise a linker sequence which mutually binds the nucleotide sequences A′ and B, wherein the linker sequence is preferably the sequence SEQ ID NO 7 (ggatatccatcacactggcggccgctcgag).

Moreover, the genetic construct according to the invention can further comprise a linker sequence SEQ ID NO 6 (ctcgagccatct) which mutually binds the sequences SEQ ID NO 1 and SEQ ID NO. 2.

As understood by a skilled person in the art, all these variations in the order of sequences included in the present document which bring about fusion proteins substantially equivalent in functional terms, must be intended as included within the scope of the present invention.

According to a preferred embodiment, the genetic construct is included inside a vector. The term vector, in the present document, means a vector known to a skilled person in the art, for example a vector chosen among plasmid, viral vector or transposon.

A further aspect of the present invention is a fusion protein comprising:
an amino acid sequence A (Cre+ ERT2) comprising an amino acid sequence SEQ ID NO. 8 related to the enzyme recombinase Cre and an amino acid sequence SEQ ID NO. 9 of a mutated receptor for estrogen ERT2; or
an amino acid sequence A′ comprising an amino acid sequence SEQ ID NO. 10 related to the fragment A of the diphtheria toxin (DTA); and
an amino acid sequence B comprising an amino acid sequence SEQ ID NO. 11 related to the inhibitor of a mutant cyclin dependent kinase (CDK) p27K⁻.

The fusion protein can further comprise a linker sequence which mutually binds the amino acid sequences A and B, wherein the linker sequence is the sequence SEQ ID NO 12 (AGYPSHWRPLEAT).

The fusion protein can further comprise a linker sequence which mutually binds the amino acid sequences A′ and B, wherein the linker sequence is the sequence SEQ ID NO 13 (SGYPSHWRPLE).

According to a preferred embodiment, the fusion protein further comprises a linker sequence SEQ ID NO 14 (LEPS) which mutually binds the sequences SEQ ID NO 8 and SEQ ID NO. 9.

A further aspect of the present invention deals with a genetic construct or a fusion protein as defined in any one of the previous aspects for use as a medicament.

A further object of the present invention deals with a genetic construct or a fusion protein as defined above for use in the tracking or ablating of quiescent cells. According to a preferred aspect, the quiescent cells are quiescent stem cells. Preferably, the quiescent cells are healthy or tumour cells.

According to a preferred aspect, it is provided to use a selective modulator of the receptor of the estrogen ERT2. Preferably, the modulator is Tamoxifene.

A further aspect of the present invention deals with a pharmaceutical composition comprising a fusion protein as defined above and Tamoxifene. Such pharmaceutical composition can further comprise one or more pharmaceutically acceptable excipients.

A further aspect of the present invention deals with a kit to be used in the tracking and/or ablating of quiescent cells comprising a fusion protein as defined above and Tamoxifene.

What is included in the present document must be intended as a non-limiting example. Moreover, the skilled person in the art will be able to understand that modifications can be performed without departing from the scope of the present invention as defined in the attached claims.

### Examples

### Experimental Part

### In Vivo Tracking of Quiescent Stem Cells of Glioma

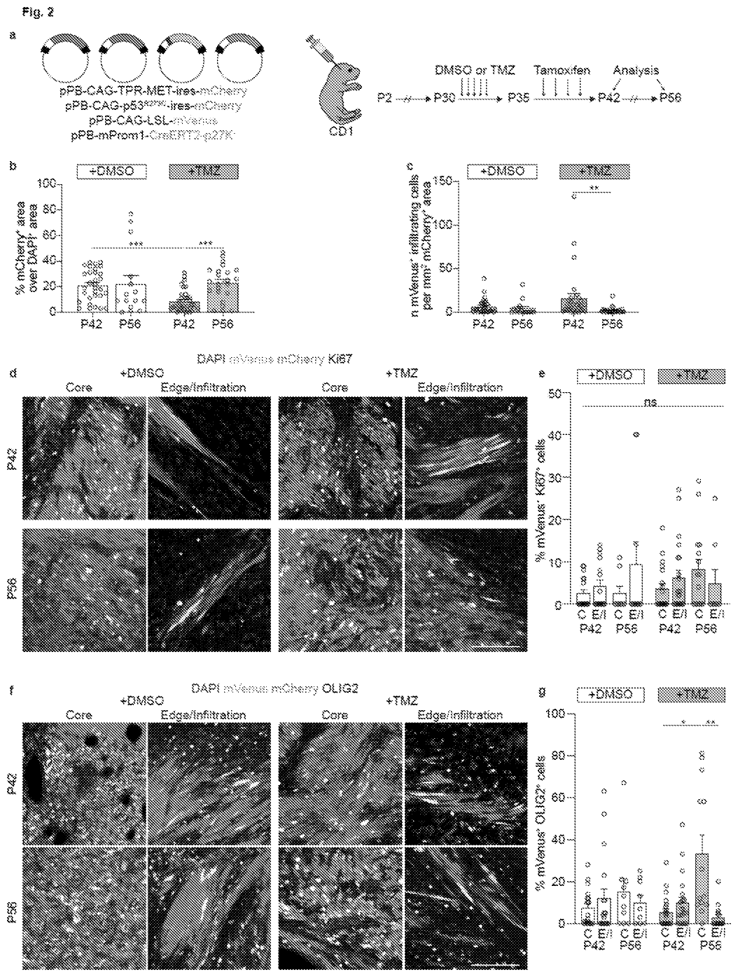

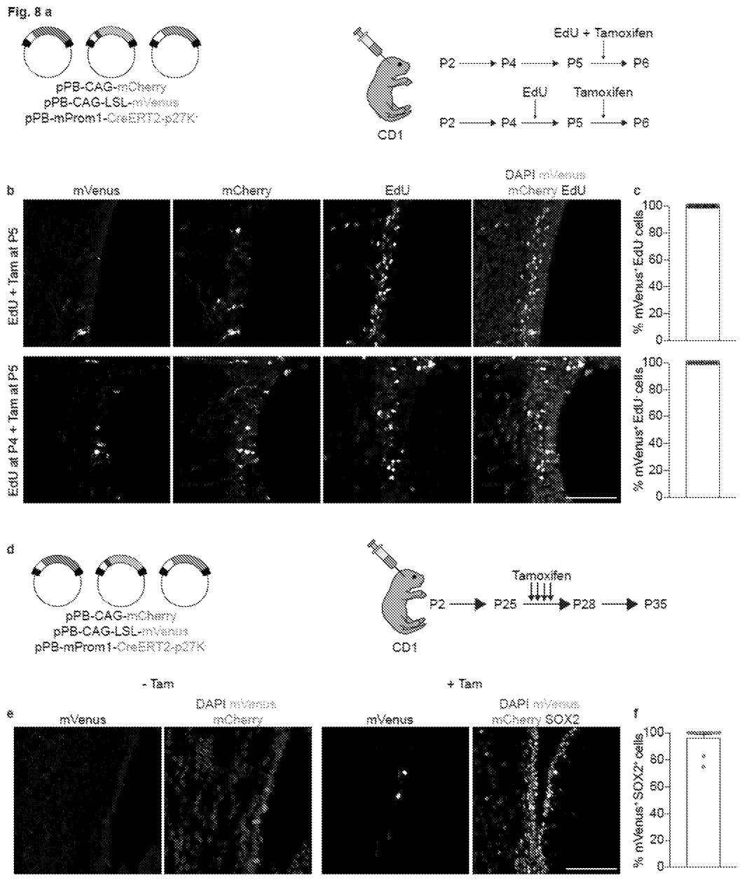

Here we have developed an inducible recombinase Cre (CreERT2) fused with p27K−, under the control of SOX2 and Prom1 promoters. The specificity of the approach has been confirmed by the labelling combined with EdU during or 1 day before the activation of the Cre through Tamoxifene (Tam), demonstrating that our new system marks the cellular progeny of the quiescent cells (FIG. 8a-c). We have also confirmed the marking of the cells Proml+ only after the injection of Tam (FIG. 8d, e). The tracking of the cellular type has shown that 96.5% of the cells derived from qProm1 was positive for SOX2 (FIG. 8f). We have analysed this system to track the cells of the progeny qProm1 in TP-induced tumours, with and without chemotherapeutic treatment. Mice have been treated with Temozolomide (TMZ) for 5 days, followed by 7 days of marking of the cells qProm1 with Tamoxifene, and then we analysed the sectioned brains (FIG. 2a). We performed the cell marking through injection of Tam after the treatment with TMZ to follow the progeny qProm1 from P42 to P56.

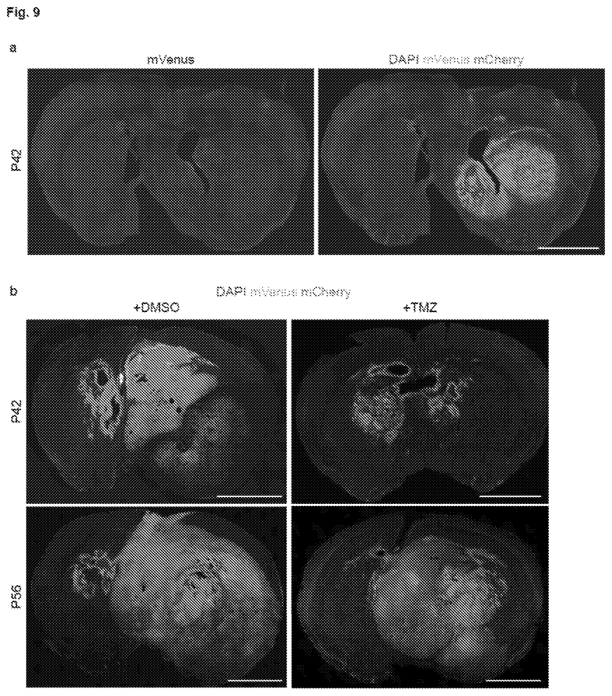

First of all, we confirmed the lack of mVenus+ with Tam absent (FIG. 9a). In order to have a neutral characterization, we analysed the whole area of the brain section measuring the brain area (DAPI+) and the tumour area (mCherry+) (FIG. 2b, Extended data FIG. 2b). Secondly, as foreseen, we have also confirmed the efficacy of the treatment with TMZ on the tumour size and the consequent rapid recurrence after chemotherapy administration (FIG. 2b). Since the capacity of the cells to infiltrate is one of the most lethal characteristics of a high-degree glioma, we analysed the number of progenies deriving from infiltrating qProm1. In mice treated with TMZ we have not observed meaningful differences with respect to the control group DMSO (FIG. 2c). Afterwards, we analysed the proliferation marker Ki67 in the cells derived from qProm1 in the core regions or the edge/infiltration regions of the tumours from P42 to P56. It is interesting to note that we have not observed meaningful differences between treatments with DMSO or TMZ (FIG. 2d, e). We have then analysed the distribution of the marker of stem cells of the glioma OLIG2 (FIG. 2f) observing a statistically meaningful increase only in the core region of the tumours of mice treated with TMZ in time (FIG. 2g). Therefore, this can suggest that the treatment with TMZ in our experimental setting has not affected the proliferation of the progeny derived from qProm1, but could affect the number of positive stem cells for OLIG2 in the tumour.

### In Vivo Ablating of Quiescent Stem Cells in the Glioma

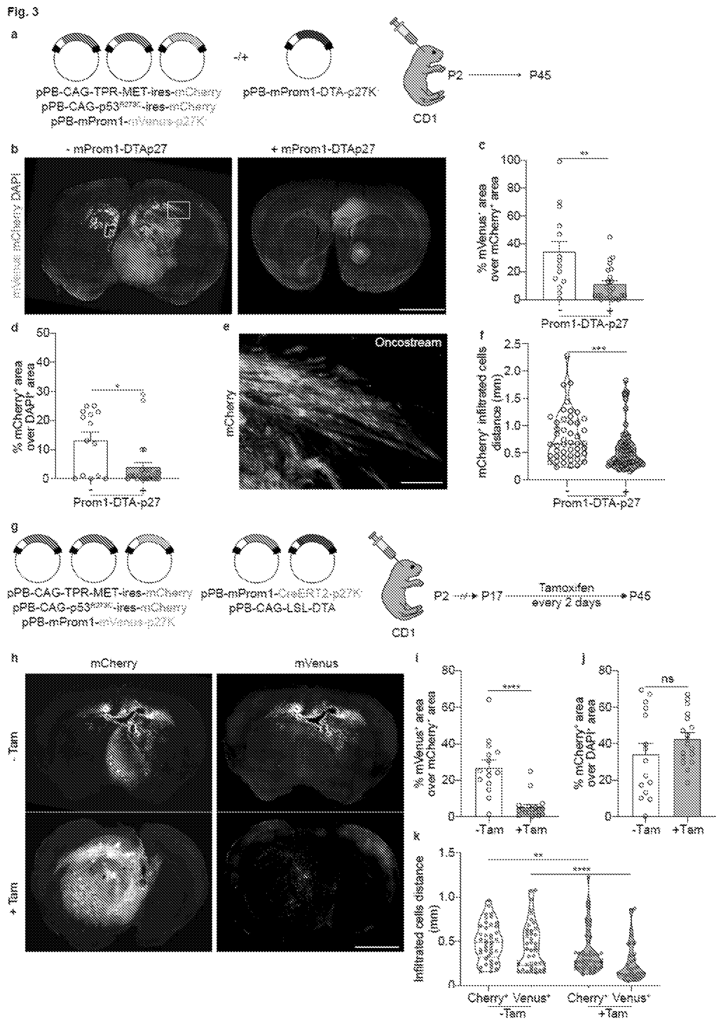

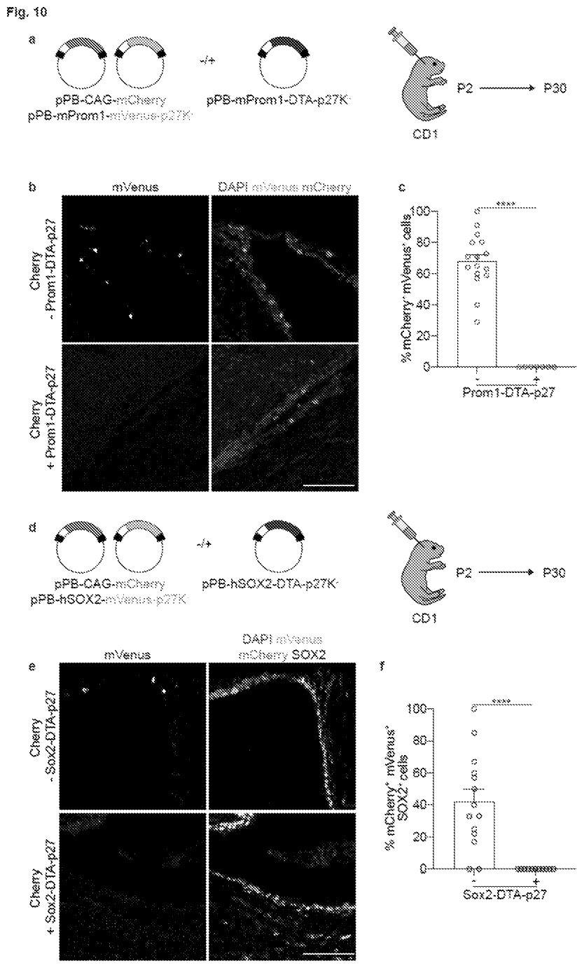

Afterwards, we tried to study the role of the cells qProm1 in the TP-mediated tumorigenesis through ablating by using the fragment A of the diphtheria toxin (DTA) fused to the isoform p27K (DTA-p27). The ablating efficiency has been verified in the neonatal area SVZ-VZ with pPB-CAG-mCherry, PB-mProml-mVenus-p27K- and pPB-mProml-DTA-p27K-(FIG. 10a), revealing the complete absence of cells qProm1 when Proml-DTA-p27 has been co-electroporated (FIG. 10b,c). We have validated the DTA-mediated ablation also in the cells qSox2 (FIG. 10d-f). Afterwards, we have verified the presence of cells qProm1 in TP-induced tumours with or without Prom1-DTA-p27 (FIG. 3a). Mice have been sacrificed at P45 and the brain sections have been histologically analysed (FIG. 3b). With respect to control mice, the co-injection of Proml-DTA-p27 has brought about the foreseen reduction of the cells qProm1 (FIG. 3c) accompanied by a smaller tumour size (FIG. 3d). As previously observed, the cells qProm1 were localized at the tumour margins, where the cells also have a shape similar to the mesenchymal/migratory one. These structures look like those which have been described as onco-stream used by the tumour cells to move, and found in samples of mice ana human tumours (FIG. 3e). The ablation of the cells qProm1 has reduced the percentage of tumour sections which showed such structures at the margins (100% in the control vs 57.14% in the group+ Proml-DTA-p27, p=0.0034). It is interesting to note that we have also observed a strong reduction of the cells infiltrated by the tumour (FIG. 3f). To study the effect of the depletion of the cells qProm1 also when the tumour is already formed and not by its genesis, we electroporated newly born mice with TP-Cherry+ mProml-Venus-p27K-together with pPB-mProml-CreERT2-p27K- and pPB-CAG-LSL-DTA (FIG. 3g). This has allowed controlling the DTA-mediated ablation of the cells qProm1 through injection of Tamoxifene from P17 to P45 (FIG. 3g). The analysis of the brain section has confirmed a reduction of the population qProm1 (FIG. 3h,i) but surprisingly the tumour size has not been affected (FIG. 3h,j) after the injection of Tamoxifene. Though the tumour growth has not been affected, the ablation of the cells qProm1 has brough about a reduction of the infiltrating cells (FIG. 3k). Taking into account the small contribution of qProm1 in the TP-induced tumorigenesis also after chemotherapy (FIG. 2), all together the tracking and ablating experiments of this cellular type have suggested that the cells qProm1 have a role in the infiltration/diffusion of the tumour cells.

### Imaging of Living Infiltrating Quiescent Stem Cells in the Organoids of Human Paediatric Brain Tumours

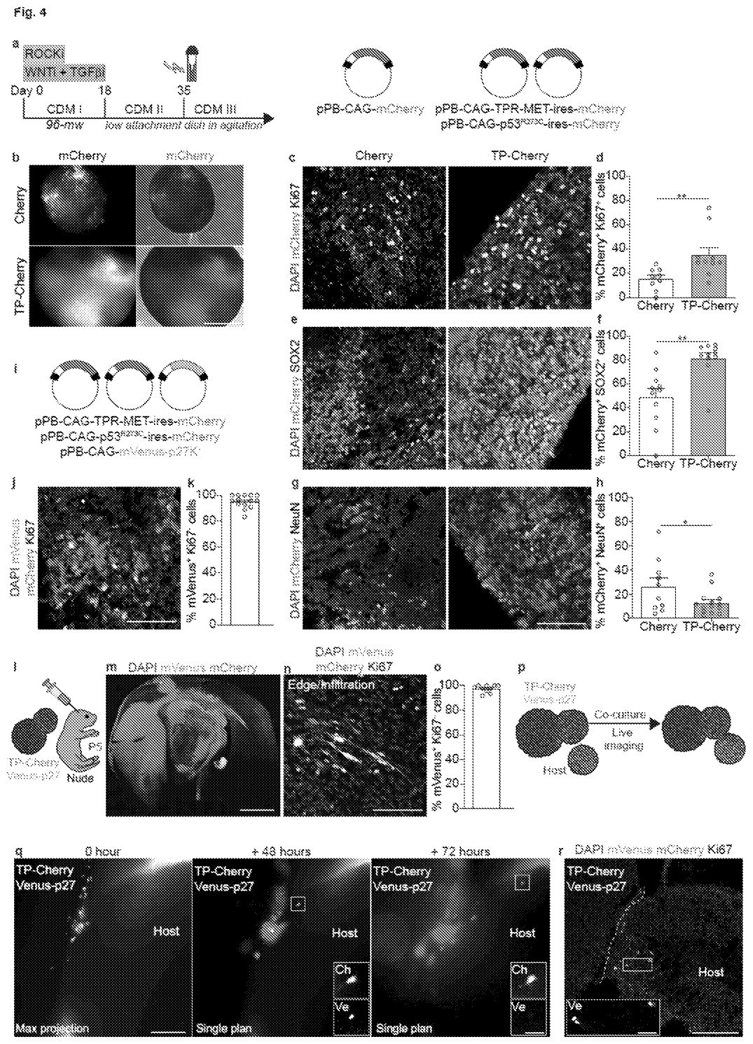

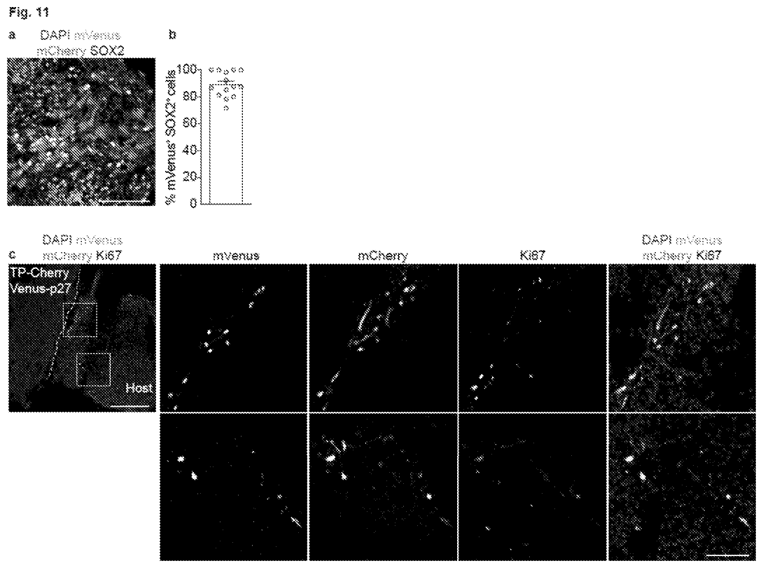

These data point out that, in brain mouse tumours, quiescent cells are necessary for the tumour invasiveness; therefore, we have validated our results also in human tumours. To reply to this question, we have developed a new model of cancer organoid of human brain. TP-Cherry (or Cherry as control) has been electroporated in organoids of the dorsal forebrain differentiated by pluripotent induced human stem cells (hiPSC) (FIG. 4a, b). In vivo imaging of organoids 30 days after electroporation (30 dpe) has shown a fluorescence mCherry+ more diffused with respect to the control (FIG. 4b). The histologic characterization of organoids has confirmed that the electroporation with TP-Cherry has brought about an increase of proliferating cells (FIG. 4c, d) and of the marker of stem cells SOX2 (FIG. 4e, f) and a reduction of the neuronal marker NeuN (FIG. 4g, h). To perform the in vivo imaging of the quiescent cells we decided to use a strong and ubiquitous promoter, such as CAG. Therefore, we afterwards expressed the quiescence sensor CAG-mVenus-p27K-(Venus-p27) together with TP-Cherry in brain organoids to visualize the cells with a slow cycle (FIG. 4i). We discovered that 89.2% of the cells mCherry+/mVenus+ co-expressed SOX2 (FIG. 11a,b) and that 95.2% of the cells mCherry+/mVenus+ was negative for the proliferation marker Ki67 (FIG. 4j,k) confirming the visualize of quiescent stem cells in the brain tumour organoids. We have further characterized the infiltration potential of those cells in vivo after transplant of electroporated organoids TP-mCherry/mVenus-p27 in the brain of nude mice (FIG. 4l-o). We then analysed the methylation profile of the DNA extracted from tumours derived from organoids transplanted in the brain of nude mice, using the brain tumour classifier, an instrument recently introduced, which improves the diagnostic process for patients affected by brain cancer. The three tested tumours were grouped in the methylation class “infantile hemispheric glioma” with a calibrated score>0.45 (score 0.73, 0.61, 0.46). Finally, we performed the in vivo imaging of TP-Cherry/Venus-p27 in co-culture with non-electroporated brain organoids (control) (FIG. 4p), which clearly revealed the infiltration of cells mCherry+/mVenus+ (FIG. 4q). We also confirmed the infiltration of cells mCherry+/mVenus+/Ki67—in the cryosections of co-cultured organoids (FIG. 4r, FIG. 11c). These data show and describe a new model based on cancer organoids for paediatric brain to study the role of quiescent cells.

### DISCUSSION

In spite of the fact that the presence of quiescent stem cells has already been demonstrated, their role in cancer infiltration remains unclear. Here, we created a new murine model of brain cancer which expressed TPR-MET and p53 mutant together with a known phase sensor of cellular cycle G0 (mVenus-p27K−). This allowed to directly visualize the quiescent cells Proml+ or Sox2+ inside the tumour and characterize their localization in core or infiltrating tumour areas. Moreover, in the infiltrating edge, they show a mesenchymal/migratory morphology with a strong accumulation of N-cadherin upon the cell-cell contact. Though it has been suggested that quiescent tumour stem cells contribute to the tumour re-growth after chemotherapy, here we have not observed an increase of the proliferation in the progeny qProm1 after the treatment with TMZ, may be due to the time window used in our study. We have not fixed a following time point, due to the high lethality of the tumour. It is interesting to note that, in our study, the treatment with TMZ increased the percentage of cells derived from qProm1 which express OLIG2 in the core areas of the tumour (FIG. 2f, g). Moreover, we have specifically ablated/removed the quiescent cells Proml+ through a new DTA degradable in phase G0 (DTA-p27K−). When ablation has been performed from the beginning of the tumour growth, this brought about a reduction of the tumour size, in addition to the infiltration and the diffusion of the tumour. On the other hand, the ablation of the cells qProm1+ after the tumour formation has had an impact only on infiltration and diffusion of tumour cells. The new system we invented specifically performs a tracking and ablating of a cellular type based on the cellular cycle and allowed us to reveal how the qProm1+ cells mainly contribute to the tumour infiltration.

Finally, we created a new type of human brain tumour organoid to visualize and study the infiltration of quiescent cells. We demonstrated that quiescent stem cells can be displayed in cancer organoids of the human brain and have an infiltration potential in vivo after injection in nude mice. Moreover, we have been able to visualize in real time quiescent tumour cells which invade a normal brain organoid in co-culture experiments. The impact of these tools resides in the chance of studying the interaction of qCSC (quiescent cancer stem cells) inside the tumour micro-environment and of having a platform to test new therapeutic strategies to block the tumour diffusion, which can be easily exploited also in other models of human and murine cancer.

### Methods

### Cloning

The hyperactive form of the transposase piggyBac (pCMV-Hahy-pBase, pPBase) has been donated by the Wellcome Sanger Institute. The plasmid pPB-CAG-MCS-ires-mCherry has been used as donor plasmid piggyBac in which to clone through PCR other coding sequences. It has been generated by amplifying the sequence IRES and the sequence coding for mCherry and then joining them through overlapping PCR. Ires-mCherry has then been cloned in the plasmid piggyBac pPB-CAG-MCS-ires-mVenus creating pPB-CAG-MCS-ires-mCherry. TPR-MET has been amplified through PCR pBABE-pure-TPR-MET (Addgene, 10902). P53 (R273C) has been cloned from pCMV-Neo-Bam-p53R273C (donated by Alberto Inga). mVenus-p27K-has been amplified through PCR from pMXs-IP-mVenus-p27K-(as a gift from Toshihiko Oki and Toshio Kitamura). The human promoter Sox2 (hSOX2) has been sub-cloned from the plasmid pGL3-SOX2 (Addgene, 101761) in the plasmid piggyBac replacing the promoter CAG (generating pPB-hSOX2). The promoter of Mouse Prom1 (mProml) has been amplified through PCR starting from genomic mouse DNA and cloned in the vector piggyBac (generating pPB-mProml). The following primers have been used to amplify the element P2 of the murine promoter Prominin1, as previously described: Fw: TTCTTTGATATCGGTACCGGTCCAATCAGTGCGCTCAGAC (SEQ ID NO. 15); Verse: TTTCTTTCTCGAAGCTTCCTCTCCGGTCCAGCTCTCCT (SEQ ID NO. 16).

CreERT2-p27K-has been generated amplifying CreERT2 (from pPB-hSynI-creER-IRES-Venus) and p27K-(da pPB-CAG-mVenus-p27K−) and then joining them through overlapping PCR. CreERT2-p27K-has then been cloned in the plasmid vector piggyBac pPB-CAG. mVenus-p27K- or CreERT2-p27K-have been cloned from the respective pPB-CAG in pPB-hSOX2 or pPB-mProml. DTA-p27K-has been generated amplifying DTA (from pDTA-TK, Addgene 22677) and p27K-(from pPB-CAG-mVenus-p27K−) and then joining them through overlapping PCR. DTA-p27K-has then been cloned in the plasmid piggyBac pPB-mProml or pPB-hSOX2. The sequences of primer used to generate the fusion proteins are listed below:

CreERT2-p27K⁻
CreERT2: Fw (NheI):
(SEQ ID NO. 17)
5′-TTCTTTGCTAGCGCCACCATGTCCAATTTACTGACCGTACA-3′;
Rev:
(SEQ ID NO. 18)
5′-CGCCAGTGTGATGGATATCCAGCTGTGGCAGGGAAACC-3′
p27K⁻ Fw:
(SEQ ID NO. 19)
5′-GGTTTCCCTGCCACAGCTGGATATCCATCACACTGGCG-3′
Rev (EcoRI):
(SEQ ID NO. 20)
5′-TTCTTTGAATTCTCATTACGTCTGGCGTCGAA-3′
DTA-p27K⁻
DTA Fw (NheI):
(SEQ ID NO. 21)
5′-TTTCTTGCTAGCGCCACCATGGATGATGTTGTTGATTCTTCTAAA
TC-3′
Rev:
(SEQ ID NO. 22)
5′-CGCCAGTGTGATGGATATCCAGATCGCCTGACACGATTTCC-3′
p27K⁻ Fw:
(SEQ ID NO. 23)
5′-GAAATCGTGTCAGGCGATCTGGATATCCATCACACTGGCG-3′
Rev (EcoRI):
(SEQ ID NO. 24)
5′-TTCTTTGAATTCTCATTACGTCTGGCGTCGAA-3′

The plasmid pPB-CAG-LSL-MCS, used as vector in which other coding sequences are cloned, has been generated by inserting a cassette loxP-STOP-loxP (LSL) between the promoter CAG and the multiple cloning sites (MCS). DTA and mVenus have been cloned through PCR respectively generating pPB-CAG-LSL-DTA and pPB-CAG-LSL-mVenus. All constructs have been verified through DNA sequencing.

### Mice Breeding

Mice have been bred in a structure for animals certified in compliance with European Guidelines. Experiments have been approved by the Italian Ministry of Health as complying with the related regulatory standards. CD1 and CD1-Nude mice have been purchased from Charles River Laboratories. Prom1CreERT2 (JAX #017743) mice have been purchased from the Jackson Laboratory. Temozolomide (Sigma-Aldrich, T2577) has been injected intraperitoneally at 82.5 mg/kg/days for five days. Tamoxifene (Alfa Aesar, J63509) has been injected intraperitoneally at 50 mg/kg/days. EdU (Life Technologies, A10044) has been injected intraperitoneally at 50 mg/kg. Mice have been sacrificed as soon as they showed signs of pain or distress (human endpoint) or otherwise mentioned in the text or in the figures.

### In Vivo Electroporation

CD1 mice at the second days after birth (P2) have been anesthetized in ice for 2 minutes, placed on a support in a stereo-taxic apparatus and injected at the following coordinates (from lambda):-1.5 D/V, + 0.8 M/L, + 1.5 A/P. The mixture of DNA has been prepared at a concentration of 5 μg/μl, with the pPBase and the donor plasmids piggyBac mixed at a ratio 1:4. 2 μl of DNA mix have been injected using a glass capillary and a micro-injector FemtoJet (Eppendorf). The electroporation of DNA has been performed with tweezers-type electrodes using the following parameters: 100 V, 50 msec/pulse, intervals of 1000 msec, 5 electric square pulses.

### Preparation of Tissues and Immunofluorescence

Mice have been perfused through intraventricular injection of paraformaldehyde (PFA) at 4%, brains have been sectioned and post-fixed for the following 24 houses. Mouse brains have afterwards been washed in PBS IX, included in agarose at 5% and sectioned using the vibratome Leica VT 1200 at a thickness of 60 μm. The brain sections have then been washed with 0.3% Triton X-100 (Sigma) in PBS 1× and permeabilized in sodium dodecyl sulphate 1× for 15 minutes. The primary antibodies have been diluted in a solution composed of C.3% of Triton X-100 and 3 of goat serum (Sigma, G6767) in PBS 1× and incubated overnight. The secondary antibodies have been incubated for 90 minutes at ambient temperature, diluted in the same solution and the cores have been coloured with 1 μg/ml of DAPI (Sigma).

The detection of EdU has been performed after immune-coloration. Briefly, after incubation with the secondary antibody, the brain sections have been post-fixed with 400 PFA for 15 minutes at ambient temperature, washed with 3% of bovine serum albumin (BSA) (Seqens IVD, 1000-70) in PBS 1× for 10 minutes and permeabilized with 0.500 Triton X-100 for 20 minutes. The sections have then been incubated for 30 minutes with a reaction mixture containing PBS 1X, CuSO4 4 mM, Alexa Fluor® 647 Azide (Life Technologies, A10277) and sodium ascorbate 100 mM (Sigma). The sections have been washed and the cores have been coloured with 1 μg/ml of DA-PI. The antibodies used for immunofluorescence are listed below:

| Antibodies | Species | Dilution | Company | Cat. N. |
|---|---|---|---|---|
| GFP | Chicken | 1:1500 | Abcam | ab13970 |
| Ki67 | Rabbit | 1:500 | Abcam | ab15580 |
| Sox2 | Rabbit | 1:500 | Abcam | ab97959 |
| Olig2 | Rabbit | 1:1500 | Sigma Aldrich | AB9610 |
| NeuN | Mouse | 1:500 | Sigma Aldrich | MAB377 |
| N-cadherin | Rabbit | 1:1000 | Abcam | Ab18203 |
| Alexa Fluor 488 goat | Goat | 1:500 | Invitrogen | A11039 |
| anti-chicken IgY | | | | |
| Alexa Fluor 546 goat | Goat | 1:500 | Invitrogen | A11030 |
| anti-mouse IgG | | | | |
| Alexa Fluor 546 goat | Goat | 1:500 | Invitrogen | A11035 |
| anti-rabbit IgG | | | | |
| Alexa Fluor 647 goat | Goat | 1:500 | Invitrogen | A21235 |
| anti-mouse IgG | | | | |
| Alexa Fluor 647 goat | Goat | 1:500 | Invitrogen | A21245 |
| anti-rabbit IgG | | | | |

### RNA Extraction and RNA Sequencing

Brains have been sectioned and tumour tissues have been accurately insulated with a fluorescent binocular microscope, frozen in liquid nitrogen and stored at −80° C. The total RNA has been insulated from the tissues with TRIzol Reagent (Invitrogen, 15596018) according to the manufacturer's instructions, Then, the RNA quality has been checked with the high-sensitivity RNA specimen by the bioanalyzer 2100 (Agilent, G2939BA) and the extracted RNA has been stored at −80° C. till the RNA-seq analysis. The sequencing has been performed on the platform HiSeq2500 Illumina using a single-end protocol with readings of length 100 bp. The sequencing readings from the FASTQ files have been aligned on the reference genome of the mouse mm10 (Ensembl Mus musculus release 102) using the transcription abundance quantifier Salmon version 1.4.0. The package R tximeta has then been used to import the transcription quantification from Salmon and perform the summary of counts at gene level. Counts have then been resized to take into account the gene length and the library size, normalized (cut mean of values M) and filtered for a low expression with the package edgeR R (PMID:19910308), converted into CPM (Counts per million) and finally the batch has been corrected with the function ComBat of the package R sva (PMID:22257669). The package stats R has then been used for performing both the analysis of the main components and the hierarchical clustering.

### Culture, Electroporation and Transplant of Brain Organoids

Human induced pluripotent stem cells (hiPSCs, donated by Domenico Delia) have been grown on a layer of Geltrex (Gibco, A14133-01), in Essential 8 Basal Medium (Gibco, A15169-01) supplemented with E8 Supplement (Gibco, A15171-01) and P/S (penicillin 100 units/ml, streptomycin 100 μg/ml, GIBCO, 15140-122). All cells were lacking mycoplasmas. iPSC have been dissociated with EDTA (Invitrogen, 15575-038) 0.5 mM, pH 8.0, to keep the clusters of cells. The organoids of the dorsal forebrain have been generated modifying a previously described protocol for the differentiation of the dorsal forebrain. In particular, the brain organoids have been cultivated in 60-mm plates not treated with tissue (Sarstedt, 82.1194.500) in a cortical differentiation medium (CDM3 or CDM4, depending on the protocol guidelines) integrated with 1% Matrigel from day 35 onwards. At day 35, the organoids have been electroporated with several combinations of plasmids as previously described. Specifically, 15-20 organoids per condition have been electroporated with 100 μg of DNA mix containing the transposase piggyBac and the donor plasmids mixed in a ration 1:4 diluted in 100 μl of Buffer 5.

For the co-culture experiment, an organoid at 30 days after electroporation (D35+ 30) has been cultivated in narrow contact with a non-electroporated organoid in a plate with 96 black wells (Ibidi, 89621). The co-cultures have been monitored and acquired every day for 1 month with a daily mean change. The organoids have been fixed overnight with PFA at 4% at D35+ 30 or D35+ 60 (for co-culture experiments), then cryo-stored with sucrose at 30% overnight and included in the Frozen Section Compound (Leica, 3801480). The organoids have been sectioned at 20 μm using the cryostat Thermo Scientific HM525 NX. The immunofluorescence has been performed as previously described for the mouse sections.

For transplant experiments, three organoids D35+ 30 have been mechanically dissociated in 200 μl of CDM3 and 8 μl of solution have been injected with a syringe Hamilton calibre 26s (80300/00). Nude mice at 5 days after birth (P5) have been anesthetized in ice for 2 minutes, placed on a support in a stereotaxic apparatus and injected at the following coordinates (from lambda):-1.5 D/V, + 1.2 M/L, + 1.5 A/P. The animals have been sacrificed as soon as they show signs of pain or distress (human endpoint).

### Extraction of Genomic DNA and Methylation Analysis

Transplanted brains have been dissected, tumours have been separated from the brain tissue through a fluorescence binocular microscope. Tumour biopsies have been frozen in liquid nitrogen and stored at −80° C. Tumour biopsies have been lysed in a lysis buffer (20 μm EDTA, 10 μm Tris, 200 μm NaCl, 0.2% Triton X-100, 100 μg/ml proteinase K, pH 8.0) for 2 hours at 37° C. The genomic DNA has been extracted with phenol-chloroform and precipitated with isopropanol. The DNA methylation profile has been performed as previously described (ref 27 of the manuscript).

Image Acquisition with a Microscope

Brain and organoid sections have been acquitted on Leica TCS Sp8 or Nikon Eclipse Ti2 equipped with a rotary disk CREST Optics X-Light V2. The co-cultivated organoids have been acquired on Nikon Eclipse Ti2 equipped with CREST Optics X-Light V2 Spinning Disk. Images are presented as projection images at maximum intensity or as a single stack Z. Images have been processed using the ImageJ software.

### Quantification of Infiltrated Area and Cells in Mouse Brain Sections

For the quantification of the area mVenus+, Cherry+ and DAPI+, whole brain sections have been acquired with Nikon Eclipse Ti2 equipped with CREST Optics X-Light V2 Spinning Disk generating mosaic-type images. A defined threshold has been applied to each separate channel to create binary images. The area measures have been limited to the threshold. At least 3 sections have been used for the brain (the numbers of analysed mice are included in the related keys of the figures). For the quantification of the cells infiltrated with mVenus+ or Cherry+, the distance has been measured between the infiltrated cells and the tumour edge for at least 6 cells and the 3 furthest cells for each section have been taken into account. All image measures have been performed with the ImageJ software.

### Statistical Analyses

All statistical analyses have been performed with the GraphPad Prism 9 software. Quantitative data have been presented as mean±s.e.m. or as violin-type graphs with median and quartiles as mentioned in the keys of the figures. Before the statistical significance test, data have been tested for the normal distribution. For normally distributed data, the Student test has been used for 2 groups. For data which do not follow a normal distribution, the non-parametric Mann-Whitney test has been used for 2 groups, with the Kruskal-Wallis test followed by the Dunn test used for more than 2 groups. The survival curve of Kaplan-Meier followed by the Log-rank (Mantel-Cox) test has been used for testing the difference in the survival of mice.

1. A genetic construct comprising:
a)—a nucleotide sequence A (Cre+ ERT2) comprising a nucleotide sequence SEQ ID NO. 1 coding for an enzyme recombinase Cre and a sequence SEQ ID NO. 2 coding for a mutated receptor for estrogen ERT2; or
a nucleotide sequence A′ comprising a nucleotide sequence SEQ ID NO. 3 coding for the fragment A of the diphtheria toxin (DTA); and
b)—a nucleotide sequence B comprising a nucleotide sequence SEQ ID NO. 4 coding for the inhibitor of a mutant cyclin dependent kinase (CDK) p27K−.

2. The genetic construct according to claim 1, further comprising a linker sequence which mutually binds the nucleotide sequences A and B, wherein the linker sequence is preferably the sequence SEQ ID NO 5.

3. The genetic construct according to claim 1, further comprising a linker sequence which mutually binds the nucleotide sequences A′ and B.

4. The genetic construct according to claim 1, further comprising a linker sequence SEQ ID NO 6 which mutually binds the sequences SEQ ID NO 1 and SEQ ID NO. 2.

5. The genetic construct according to claim 1, wherein said construct is included inside a vector chosen among plasmid, viral vector, transposon.

6. A fusion protein comprising:
a)—an amino acid sequence A (Cre+ ERT2) comprising an amino acid sequence SEQ ID NO. 8 related to the enzyme recombinase Cre and an amino acid sequence SEQ ID NO. 9 of a mutated receptor for estrogen ERT2; or
an amino acid sequence A′ comprising an amino acid sequence SEQ ID NO. 10 related to the fragment A of the diphtheria toxin (DTA); and
b)—an amino acid sequence B comprising an amino acid sequence SEQ ID NO. 11 related to the inhibitor of a mutant cyclin dependent kinase (CDK) p27K−.

7. The fusion protein according to claim 6, further comprising a linker sequence which mutually binds the amino acid sequences A and B, wherein the linker sequence is the sequence SEQ ID NO 12.

8. The fusion protein according to claim 6, further comprising a linker sequence which mutually binds the amino acid sequences A′ and B, wherein the linker sequence is the sequence SEQ ID NO 13.

9. The fusion protein according to claim 6, further comprising a linker sequence SEQ ID NO 14 which mutually binds the sequences SEQ ID NO 8 and SEQ ID NO. 9.

10. (canceled)

11. A method for the tracking of quiescent cells, comprising providing to the quiescent cells the genetic construct according to claim 1, said genetic construct comprising:
a)—nucleotide sequence A (Cre+ ERT2) comprising a nucleotide sequence SEQ ID NO. 1 coding for an enzyme recombinase Cre and a sequence SEQ ID NO. 2 coding for a mutated receptor for estrogen ERT2; and
b)—a nucleotide sequence B comprising a nucleotide sequence SEQ ID NO. 4 coding for the inhibitor of a mutant cyclin dependent kinase (CDK) p27K
and a selective modulator of the receptor of the estrogen ERT2 and thereafter tracking the quiescent cells.

12. A method for ablating quiescent cells, comprising providing the quiescent cells with the genetic construct according to claim 1, the genetic construct comprising:
a)—nucleotide sequence A′ comprising a nucleotide sequence SEQ ID NO. 3 coding for the fragment A of the diphtheria toxin (DTA); and
b)—a nucleotide sequence B comprising a nucleotide sequence SEQ ID NO. 4 coding for the inhibitor of a mutant cyclin dependent kinase (CDK) p27K
and thereafter ablating the quiescent cells.

13. The method of claim 11, wherein the quiescent cells are quiescent stem cells.

14. The method of claim 11, wherein the quiescent cells are healthy or tumour cells.

15. The method of claim 11, wherein the modulator is Tamoxifene.

16. A composition comprising a fusion protein according to claim 6 and Tamoxifene.

17. (canceled)

18. A kit for use in tracking and/or ablating quiescent cells comprising a fusion protein according to claim 6 and Tamoxifene.

19. The genetic construct according to claim 2, wherein the linker sequence is SEQ ID NO 5.

20. The genetic construct according to claim 4, wherein the linker sequence is SEQ ID NO 7.

21. A method for the tracking of quiescent cells comprising providing to the quiescent cells a fusion protein comprising:
a)—an amino acid sequence A (Cre+ ERT2) comprising an amino acid sequence SEQ ID NO. 8 related to the enzyme recombinase Cre and an amino acid sequence SEQ ID NO. 9 of a mutated receptor for estrogen ERT2; and
b)—an amino acid sequence B comprising an amino acid sequence SEQ ID NO. 11 related to the inhibitor of a mutant cyclin dependent kinase (CDK) p27K− and a selective modulator of a receptor of the estrogen ERT2 and thereafter tracking the quiescent cells.

22. A method for ablating quiescent cells, comprising providing the quiescent cells with the fusion protein according to claim 8 comprising:
a)—an amino acid sequence A′ comprising an amino acid sequence SEQ ID NO. 10 related to the fragment A of the diphtheria toxin (DTA); and
b)—an amino acid sequence B comprising an amino acid sequence SEQ ID NO. 11 related to the inhibitor of a mutant cyclin dependent kinase (CDK) p27K− and thereafter ablating the quiescent cells.
